(19) United States
(12) Patent Application Publication
(10) Pub. No.: US 2026/0051373 A1
(43) Pub. Date: Feb. 19, 2026

(54) OPTIMIZATION OF CONCRETE MIXES AND PERFORMANCE ANALYSIS USING ARTIFICIAL INTELLIGENCE

(71) Applicant: Saudi Arabian Oil Company, Dhahran (SA)

(72) Inventors: Zakariya Saleh Al-Helal, Dhahran (SA); Tarik Hoshan, Dhahran (SA); Carlos Ernesto Acero, Dhahran (SA); Ibrahim Al Tarouti, Al Qatif (SA)

(73) Assignee: Saudi Arabian Oil Company, Dhahran (SA)

(21) Appl. No.: 18/802,267

(22) Filed: Aug. 13, 2024

(51) Int. Cl. G16C 60/00; G16C 20/30; G16C 20/70

(52) U.S. Cl. CPC G16C 60/00; G16C 20/30; G16C 20/70

(57) ABSTRACT

An artificial intelligence (AI)-based system for optimizing economic, environmental, and technical performance of concrete mixes. The system may obtain concrete mix data from laboratory testing of trial concrete mixes and production concrete mixes. The system may also obtain results from experiments designed to determine the impact of variation in raw materials. An AI model may be trained using the concrete mix data, and the trained AI model may be used to determine an optimize concrete mix having specific chemical and mechanical properties, environmental impacts, and financial performance. The trained AI model may be used to certify concrete mixes, provide performance metrics of laboratories testing concrete mixes, analyze performance of the trial and production concrete mixes, determine environmental product declarations, and analyze raw materials in concrete mixes in addition to provisioning geographic information system (GIS) information.

### BACKGROUND

### Field of the Disclosure

The present disclosure generally relates to the monitoring of production of concrete. More specifically, embodiments of the disclosure relate to the determination of performance of concrete mixes, and the determination and production of optimal concrete mixes, environmental certification of concrete production, monitoring of laboratories performance, and provision of raw materials analytics.

### Description of the Related Art

Concrete is commonly used throughout the world as a building material because of its mechanical properties and widespread availability. Concrete may be formed from various combinations of cement, water, sand, aggregate, and additives. The properties of concrete, such as the compressive strength and tensile strength, may be altered by varying the ratios of raw materials (that is, “ingredients”) and including additives. Additionally, the environmental impact of concrete is also a function of the amounts and types of ingredients. Determining the optimal ratios and amounts of concrete ingredients for application and environmental requirements may be challenging.

### SUMMARY

Embodiments of the disclosure related to an artificial intelligence (AI)-based optimization of the economic, environmental, and technical performance of concrete mixes.

In one embodiment, a method for determining a concrete mix composition is provided. The method includes obtaining a plurality of concrete mix compositions and respective mix properties, the respective mix properties including compressive strength, slump measurements, temperature measurements, tensile strength measurements, flexural strength measurements, elastic modulus measurements, Poisson's ratio measurements, creep measurements, chloride penetration measurements, permeability measurements or a combination thereof, such that the plurality of concrete mix compositions and respective mix properties are obtained from respective laboratory tests of the plurality of concrete mix compositions, respective batch plant tests of the plurality of concrete mix compositions, or a combination thereof, such that the plurality of concrete mix compositions include a production concrete mix composition and a trial concrete mix composition. The method also includes processing the plurality of concrete mix compositions and respective mix properties to obtain a training dataset and testing dataset, training a concrete mix artificial intelligence (AI) model using the plurality of concrete mix compositions and respective mix properties of the training dataset to create a concrete mix AI model, and using the trained concrete mix AI model to determine an optimized concrete mix composition having a specific set of mix properties.

In some embodiments, the method includes obtaining a plurality of environmental impact parameters associated with the plurality of concrete mix compositions, processing the plurality of concrete mix compositions and environmental impact parameters into the training dataset and the testing dataset, and training the artificial intelligence (AI) using the plurality of concrete mix compositions and environmental impact parameters of the training dataset to create the concrete mix AI model. In such embodiments, the method includes using the trained concrete mix AI model to determine an environmental product declaration (EPD) of one of the plurality of concrete mix compositions or the optimized concrete mix composition. In some embodiments, using the trained concrete mix AI model to determine an optimized concrete mix composition having a specific set of mix properties includes using the trained concrete mix AI model to determine an optimized concrete mix composition optimized for a raw material, such that the optimization for the raw material includes environmental impact, technical performance, mix properties, or a combination thereof. In some embodiments, the concrete mix AI model uses an artificial neural network (ANN), fuzzy logic, a genetic algorithm, a hybrid AI model, a genetic weighted pyramid operation tree, or a combination thereof. In some embodiments, the method includes using the trained concrete mix AI model to determine a performance metric of the respective laboratory tests or the respective batch plant tests. In some embodiments, the method includes using the trained concrete mix AI model to determine the quality, the production, or a combination thereof of the plurality of concrete mix compositions from the respective laboratory tests or the respective batch plant tests. In some embodiments, the method includes providing an output from the AI model to a geographic information system (GIS) to monitor use of a raw material at a location in the GIS. In some embodiments, the method includes obtaining a plurality of experiment results, each of the experiment results associated with an experiment designed to test a raw material used in concrete mixes, processing the plurality of experiment results into the training dataset and the testing dataset, and training the artificial intelligence (AI) using the plurality of experiment results of the training dataset to create the concrete mix AI model. In some embodiments, the method includes providing the optimized concrete mix composition to a concrete batch plant and modifying a mixing process or a concrete mix composition of the concrete batch plant to produce the optimized concrete mix composition.

In another embodiment, a non-transitory computer readable storage medium comprising program instructions stored thereon for determining a concrete mix composition is provided. The program instructions are executable by a processor to perform operations that include obtaining a plurality of concrete mix compositions and respective mix properties, the respective mix properties including compressive strength, slump measurements, temperature measurements, tensile strength measurements, flexural strength measurements, elastic modulus measurements, Poisson's ratio measurements, creep measurements, chloride penetration measurements, permeability measurements or a combination thereof, such that the plurality of concrete mix compositions and respective mix properties are obtained from respective laboratory tests of the plurality of concrete mix compositions, respective batch plant tests of the plurality of concrete mix compositions, or a combination thereof, such that the plurality of concrete mix compositions include a production concrete mix composition and a trial concrete mix composition. The operations also include processing the plurality of concrete mix compositions and respective mix properties to obtain a training dataset and testing dataset, training a concrete mix artificial intelligence (AI) model using the plurality of concrete mix compositions and respective mix properties of the training dataset to create a concrete mix AI model, and using the trained concrete mix AI model to determine an optimized concrete mix composition having a specific set of mix properties.

In some embodiments, the operations also include obtaining a plurality of environmental impact parameters associated with the plurality of concrete mix compositions, processing the plurality of concrete mix compositions and environmental impact parameters into the training dataset and the testing dataset, and training the artificial intelligence (AI) using the plurality of concrete mix compositions and environmental impact parameters of the training dataset to create the concrete mix AI model. In such embodiments, the operations include using the trained concrete mix AI model to determine an environmental product declaration (EPD) of one of the plurality of concrete mix compositions or the optimized concrete mix composition. In some embodiments, using the trained concrete mix AI model to determine an optimized concrete mix composition having a specific set of mix properties includes using the trained concrete mix AI model to determine an optimized concrete mix composition optimized for a raw material, such that the optimization for the raw material includes environmental impact, technical performance, mix properties, or a combination thereof. In some embodiments, the concrete mix AI model uses an artificial neural network (ANN), fuzzy logic, a genetic algorithm, a hybrid AI model, a genetic weighted pyramid operation tree, or a combination thereof. In some embodiments, the operations include using the trained concrete mix AI model to determine a performance metric of the respective laboratory tests or the respective batch plant tests. In some embodiments, the operations include using the trained concrete mix AI model to determine the quality, the production, or a combination thereof of the plurality of concrete mix compositions from the respective laboratory tests or the respective batch plant tests. In some embodiments, the operations include providing an output from the AI model to a geographic information system (GIS) to monitor use of a raw material at a location in the GIS. In some embodiments, the operations include obtaining a plurality of experiment results, each of the experiment results associated with an experiment designed to test a raw material used in concrete mixes, processing the plurality of experiment results into the training dataset and the testing dataset, and training the artificial intelligence (AI) using the plurality of experiment results of the training dataset to create the concrete mix AI model. In some embodiments, the operations include providing the optimized concrete mix composition to a concrete batch plant and modifying a mixing process or a concrete mix composition of the concrete batch plant to produce the optimized concrete mix composition.

In another embodiment, a system for determining a concrete mix composition is provided. The system includes a processor and a non-transitory computer-readable memory accessible by the processor and having executable code stored thereon. The executable code includes a set of instructions that causes the processor to perform operations that include obtaining a plurality of concrete mix compositions and respective mix properties, the respective mix properties including compressive strength, slump measurements, temperature measurements, tensile strength measurements, flexural strength measurements, elastic modulus measurements, Poisson's ratio measurements, creep measurements, chloride penetration measurements, permeability measurements or a combination thereof, such that the plurality of concrete mix compositions and respective mix properties are obtained from respective laboratory tests of the plurality of concrete mix compositions, respective batch plant tests of the plurality of concrete mix compositions, or a combination thereof, such that the plurality of concrete mix compositions include a production concrete mix composition and a trial concrete mix composition. The operations also include processing the plurality of concrete mix compositions and respective mix properties to obtain a training dataset and testing dataset, training a concrete mix artificial intelligence (AI) model using the plurality of concrete mix compositions and respective mix properties of the training dataset to create a concrete mix AI model, and using the trained concrete mix AI model to determine an optimized concrete mix composition having a specific set of mix properties.

In some embodiments, the operations also include obtaining a plurality of environmental impact parameters associated with the plurality of concrete mix compositions, processing the plurality of concrete mix compositions and environmental impact parameters into the training dataset and the testing dataset, and training the artificial intelligence (AI) using the plurality of concrete mix compositions and environmental impact parameters of the training dataset to create the concrete mix AI model. In such embodiments, the operations include using the trained concrete mix AI model to determine an environmental product declaration (EPD) of one of the plurality of concrete mix compositions or the optimized concrete mix composition. In some embodiments, using the trained concrete mix AI model to determine an optimized concrete mix composition having a specific set of mix properties includes using the trained concrete mix AI model to determine an optimized concrete mix composition optimized for a raw material, such that the optimization for the raw material includes environmental impact, technical performance, mix properties, or a combination thereof. In some embodiments, the concrete mix AI model uses an artificial neural network (ANN), fuzzy logic, a genetic algorithm, a hybrid AI model, a genetic weighted pyramid operation tree, or a combination thereof. In some embodiments, the operations include using the trained concrete mix AI model to determine a performance metric of the respective laboratory tests or the respective batch plant tests. In some embodiments, the operations include using the trained concrete mix AI model to determine the quality, the production, or a combination thereof of the plurality of concrete mix compositions from the respective laboratory tests or the respective batch plant tests. In some embodiments, the operations include providing an output from the AI model to a geographic information system (GIS) to monitor use of a raw material at a location in the GIS. In some embodiments, the operations include obtaining a plurality of experiment results, each of the experiment results associated with an experiment designed to test a raw material used in concrete mixes, processing the plurality of experiment results into the training dataset and the testing dataset, and training the artificial intelligence (AI) using the plurality of experiment results of the training dataset to create the concrete mix AI model. In some embodiments, the operations include providing the optimized concrete mix composition to a concrete batch plant and modifying a mixing process or a concrete mix composition of the concrete batch plant to produce the optimized concrete mix composition.

### BRIEF DESCRIPTION OF THE DRAWINGS

FIG. 1 is a block diagram of a process for optimization of concrete mixes and performance analysis using artificial intelligence (AI) in accordance with an embodiment of the disclosure;

FIG. 2 is a block diagram of a process for the development and use of a concrete mix AI model in accordance with an embodiment of the disclosure; and

FIG. 3 is a block diagram of a block diagram of an AI-based system for the optimization of concrete mixes and performance analysis in accordance with an embodiment of the disclosure.

### DETAILED DESCRIPTION

The present disclosure will be described more fully with reference to the accompanying drawings, which illustrate embodiments of the disclosure. This disclosure may, however, be embodied in many different forms and should not be construed as limited to the illustrated embodiments. Rather, these embodiments are provided so that this disclosure will be thorough and complete, and will fully convey the scope of the disclosure to those skilled in the art.

Embodiments of the disclosure are directed to an artificial intelligence (AI)-based system for optimizing economic, environmental, and technical performance of concrete mixes. The system may obtain concrete mix data from the supply chain (for example, from laboratories that analyze concrete mixes from concrete producers) and from concrete mix trials (for example, from laboratories that test and analyze trial concrete mixes). The AI system may also receive data from design experiments that provide design data of non-production mixes. The AI system may produce designs for optimized concrete mixes that conform to certain requirements, generate environmental analysis of concrete mixes, provide analytics about raw materials, and monitor the performance of concrete mixes produced by the concrete supply chain and trial laboratories.

Advantageously, embodiments of the disclosure provide connectivity to the supply chain and may receive a continuous input of concrete mix data from the supply chain and from testing of trial concrete mixes. Embodiments of the disclosure also provide monitoring of the performance of concrete suppliers and testing laboratories by validating the quality of the laboratories'data output (the input provided to the AI-based system). Moreover, embodiments of the disclosure may reduce the cost, reduce carbon dioxide (CO₂) emissions, and improve the technical performance of concrete mixes.

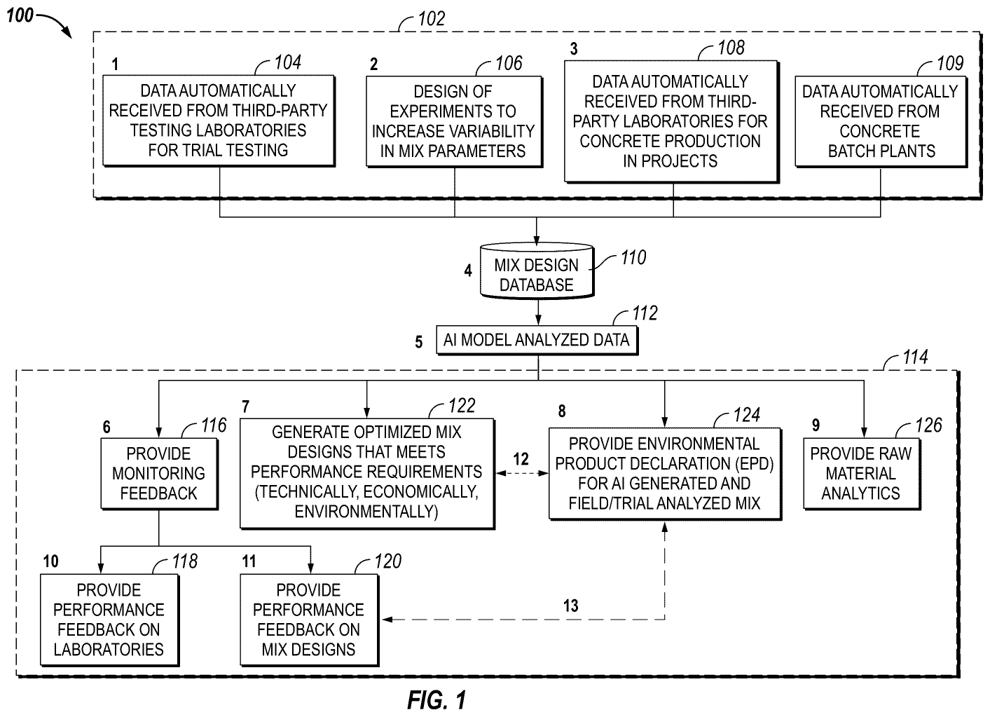

FIG. 1 depicts a process 100 for optimization of concrete mixes and performance analysis using artificial intelligence (AI) in accordance with an embodiment of the disclosure. As shown in FIG. 1, the process 100 may receive inputs 102. The inputs 102 may include data from third-party testing laboratories for concrete mix trials (block 104), data from experiments designed to increase the variability of mix parameters (block 106), data received from third party testing laboratories for concrete production mixes in use in projects (block 108), and data received from batch plants (that is, concrete producers) for concrete mixes (block 109). In some embodiments, the inputs 102 may be obtained as a “live feed” directly from third-party laboratories or producers via an interconnected computer network (for example, the Internet or an intranet). The data 102 may be stored in an organized collection of data, such as a mix design database 110.

The concrete mix trials data (block 104) may include performance reports that include the performance of concrete mixes tested in laboratory trials. The concrete production mix data (block 108) and batch plants data may include performance reports of concrete mixes produced or currently in use in construction projects (for example, as building materials). The performance reports may include, for example, concrete compressive strength reports that provide the compressive strength measurements and the composition of an associated concrete mix. In some embodiments, the performance reports may additionally or alternatively include other mechanical or chemical properties (referred to herein as “mix properties”) that measure the performance of an associated concrete mix, including but not limited to: slump measurements, temperature measurements, tensile strength measurements, flexural strength measurements, elastic modulus measurements, Poisson's ratio measurements, creep measurements, chloride penetration measurements, permeability measurements, and any combination thereof. In some embodiments, the performance reports may also include the environmental impact of a concrete mix. The environmental impact parameters may include but are not limited to: total energy consumption, concrete water use (batch and wash), global warming potential, ozone depletion, acidification, eutrophication, petrochemical ozone creation, and any combination thereof.

The design experiment data (block 106) may include performance data for concrete mixes outside the range of normal production mixes to determine the impact of variation in mix parameters and raw materials. Such raw materials may include, for example, cement, water, coarse aggregates (for example, gravel and crushed stone), fine aggregates (for example, sand), additives (for example, plasticizers, accelerators, and retarders), and supplementary cementitious materials (for example, natural pozzolan, fly ash, slag cement and silica fume). This may include, for example, experiments designed to use a maximum amount of a concrete mix raw material that would be impractical in a production use in order to determine the effect of the raw material on mechanical or chemical properties of the concrete mix, the effect of the raw material on an environmental impact of the concrete mix. The mechanical properties affected by a raw material may include but are not limited to: compressive strength, tensile strength measurements, flexural strength measurements, elastic modulus measurements, Poisson's ratio measurements, creep measurements, slump measurements, and any combination thereof. The chemical properties affected by a raw material may include but are not limited to: chloride penetration measurements, permeability measurements and any combination thereof. The environmental impact parameters affected by a raw material may include but are not limited to: total energy consumption, concrete water use (batch and wash), global warming potential, ozone depletion, acidification, eutrophication, petrochemical ozone creation, and any combination thereof. In another example, the design experiment data may include experiments designed to maximize the combination of technical and environmental performance of a concrete mix; the resulting concrete mix may not achieve the overall optimal technical performance or environmental performance but an optimization between both objectives.

Next, an artificial intelligence (AI) model may analyze the data (block 112) stored in the mix design database 110. Various outputs 114 may be produced by the analysis performed by the AI model. In some embodiments, the AI model may provide monitoring feedback (block 116). In some embodiments, providing the monitoring feedback (block 116) may include providing performance feedback (for example, metrics) on the laboratories or batch plants (block 118). In some embodiments, the performance of a technician conducting laboratory testing or batch plant testing may be determined. For example, the performance feedback on laboratories may include an indicator of technician competency in performing concrete testing or the consistency of a concrete supply. In some embodiments, the performance feedback may include cross-checking the accuracy of one laboratory or batch plant to another laboratory or batch plant, or cross-checking the accuracy of one supplier to another supplier. The cross-checking may enable the validation of reported parameters. In some embodiments, providing the monitoring feedback may include providing performance feedback on concrete mix designs (block 120). The performance feedback may include the mechanical or chemical properties of a concrete mix.

In some embodiments, the AI model may generate optimized concrete mix designs (that is, compositions) that meet certain performance requirements (block 122). For example, the AI model may provide a concrete mix designs having certain mechanical properties, chemical properties, environmental impacts, financial impacts, or a combination thereof. In some embodiments, an optimized concrete mix design may be provided to a concrete batch plant that may use the optimized concrete mix design to modify the batch plan mixing system or concrete mix composition according to the proportions of raw materials specified by the optimized concrete mix design. In such embodiments, the concrete batch plant may receive the optimized concrete mix design directly and without user intervention (for example, via communications network) to automatically modify the batch plant mixing system to produce concrete mixes according to the optimized concrete mix design.

In some embodiments, the AI model may include or provide information to modules that optimize concrete mixes for different countries or regions based on the availability of raw materials. For example, a country or region that does not have access to a particular raw material may only receive an optimized concrete mix design that omits that particular raw material and that may include a suitable substitute. In some embodiments, the output from the AI model may be provided to a geographic information system (GIS) to monitor the use of a raw material at a location in the GIS. The optimization for a raw material may be based on environmental impact, technical performance, mix properties, or a combination thereof. In some embodiments, a technical parameter or raw material for optimization (that is, either maximization or minimization) may be provided as a user-selectable input; in such embodiments, the AI model may receive the user input and determine an optimized concrete mix using the selected technical parameter or raw material. For example, a user may select to maximize the use of Pozzolan in a concrete mix.

In some embodiments, the AI model may provide an environmental product declaration (EPD) for AI-generated mixes and analyzed trial and field concrete mixes provided by laboratories or batch plants (block 124). In some embodiments, an EPD may be generated for a specific concrete mix, or an EPD may be generated for the average of concrete mixes for a supplier's production performance. For example, an EPD may include water and cement use in concrete. In some embodiments, an EPD may include but is not limited to: carbon dioxide (CO₂) emissions, carbon dioxide equivalent (CO₂e) emissions, total energy consumption, concrete water use (batch and wash), global warming potential, ozone depletion, acidification, eutrophication, petrochemical ozone creation, and any combination thereof. In some embodiments, an EPD may include mechanical or chemical properties of a concrete mix, such as compressive strength, slump, or a combination thereof.

In some embodiments, the AI model may provide an analysis of raw materials used in the concrete mixes (block 126). The analysis may include the effect of raw materials on the performance of the concrete. This may enable the optimization of the use of raw materials based on engineering requirements and source location. For example, the analysis may include a concentration of a raw material and its effect on a mechanical property of a concrete mix, alone or in combination with other raw materials. In another example, the analysis may include concentration of a raw material and its effect on an environmental impact of a concrete mix. In another example, the analysis may provide information about the characteristics of the raw materials from different sources. The analysis may also be used to value engineer the design requirements of new mixes (for example, a current requirement of cement content may get changed by the engineering team for future mixes because of insights and information from the analysis information.

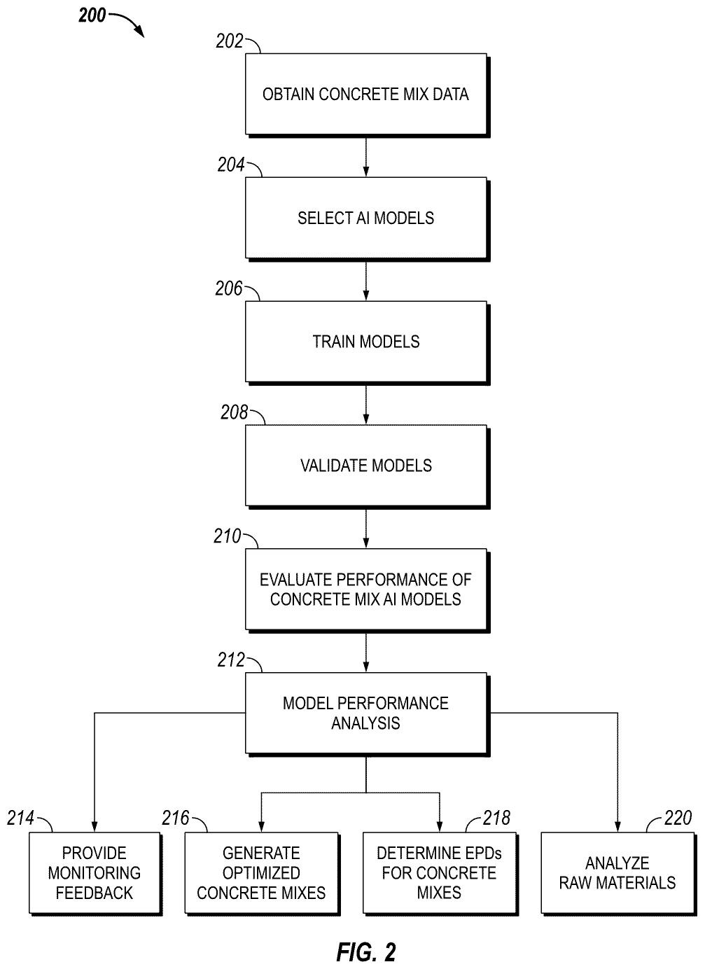

FIG. 2 depicts a process 200 for the development and use of a concrete mix AI model in accordance with an embodiment of the disclosure. Initially, concrete mix data may be obtained (block 202) from the data sources discussed supra, such as third party laboratories providing performance data of trial concrete mixes and produced concrete mixes from concrete producers. Additionally, the concrete mix data may include experimental data from experiments designed to test other concrete mixes or impact of a mix ingredient.

Next, one or more AI models may be selected (block 204) for training and use with the concrete mix data. The one or more AI models may include an artificial neural network (ANN), a fuzzy logic model, a genetic algorithm, a hybrid AI model, and a genetic weighted pyramid operation tree, or other suitable AI models. Selection of an AI model (or a combination of AI models) may also include selection of the design and parameters of the AI model. For example, for an ANN, the selection may include determining the number of layers, number of neurons in each layer, activation functions, and other parameters of the neural network architecture. In another example, for a fuzzy logic model, the selection may include defining fuzzy sets and membership functions for each input variable based on expert knowledge or data-driven techniques.

The selected AI models may be trained using the concrete mix data (block 206). For example, a percentage of the concrete mix data may be used for training the selected AI models, while the remaining concrete mix data may be used for validation, for actual performance analysis, or both. The training data is provided as input to the selected AI models. The training may also include adjusted parameters of the AI models based on AI optimization techniques (e.g., loss function minimization).

In some embodiments, the trained AI models may be validated (that is, tested) using the concrete mix data (block 208). As mentioned supra, a selected percentage of the concrete mix data may be used for validation of the trained AI models. Next, the performance of the validated concrete mix models may be evaluated based on the validation/testing data (block 210). The evaluation may include metrics such as accuracy, precision, recall, F1-score, or other suitable metrics or combination thereof. Finally, one or more of the AI models may be used in a performance analysis of concrete mixes (block 212). The AI model used in the performance analysis may be selected based on the evaluation. The performance analysis may use some of the previously obtained concrete mix data (for example, concrete mix data that was not selected for training and testing) or using newly obtained concrete mix data. Additionally, as discussed supra, the concrete mix AI model may be used to provide monitoring feedback (block 214), generate optimized concrete mixes that meet certain requirements (block 216), determine EPDs for concrete mixes (block 218), and analyze raw materials used in concrete mixes (block 220).

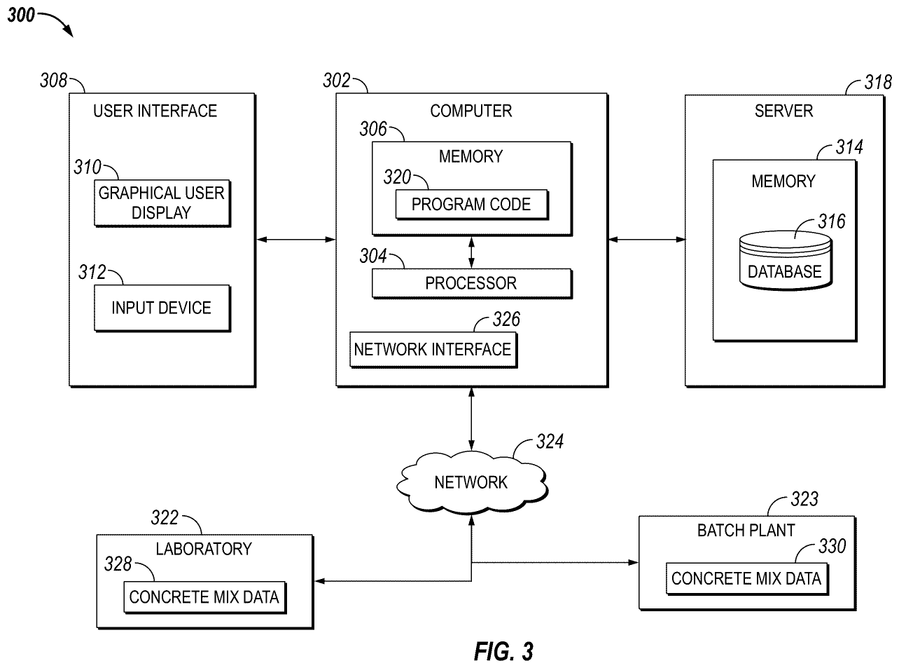

FIG. 3 is a block diagram of an AI-based system 300 for the optimization of concrete mixes and performance analysis in accordance with an embodiment of the disclosure. As shown in FIG. 3, the AI-based system may include a computer 302, discussed further infra. The computer 302 may be a computer of any type of suitable processing capacity, such as a personal computer, laptop computer, tablet computer, or any other suitable processing apparatus. The computer 302 may also be representative of resources available in a computer cluster or a cloud-computing platform. It should thus be understood that a number of commercially available data processing systems and types of computers may be used for this purpose.

The computer 302 may include a processor 304 and a memory 306 coupled to the processor 304 to store operating instructions, control information, and access database records therein in accordance with an embodiment of the disclosure. The processor 304 may be a multicore processor with nodes such as those from Intel Corporation or Advanced Micro Devices (AMD). The processor 304 may be or include a reduced instruction set (RISC) processor, such as a processor based on an ARM architecture. The AI-based system 300 may also be a mainframe computer of any conventional type of suitable processing capacity such as those available from International Business Machines (IBM) of Armonk, N.Y., or other source, or an HPC Linux cluster computer.

The computer 302 may include or be accessible to operators or users through user interface 308, which may receive user inputs and is available for displaying output data or records of processing results obtained according to the present disclosure with an output display 310. The output display 310 may include components such as a printer and an output display screen capable of providing printed output information or visible displays in the form of graphs, data sheets, graphical images, data plots and the like as output records or images.

The user interface 308 of computer 302 also includes a suitable user input device or input/output control unit 312 to provide a user access to control or access information, provide inputs, and operate the computer 302. The AI-based system 300 may include a database of concrete mix data in computer memory. In some embodiments, the databased may be stored in internal memory 306. In other embodiments, as shown in FIG. 3, the database may be an associated database 316 stored in a memory 314 of a server 318 accessible by the computer 302 via communications network (now shown).

The AI-based system 300 includes executable code 320 stored in the non-transitory memory 306 of the computer 302. The executable code 320 according to the present disclosure is in the form of computer operable instructions causing the data processor 304 to receive input data and provide outputs based on processing the input data. The computer operable instructions of the executable code 320 may execute and train a concrete mix AI model according to the techniques described herein, and may generate a concrete mix design using the trained concrete mix AI model.

The executable code 320 may be in the form of microcode, programs, routines, or symbolic computer operable languages capable of providing a specific set of ordered operations controlling the functioning of the AI-based system 300 and direct its operation. The instructions of executable code 320 may be stored in memory 306 of the AI-based system 300, or on computer diskette, magnetic tape, conventional hard disk drive, electronic read-only memory, optical storage device, solid-state storage, or other appropriate data storage device having a non-transitory computer readable storage medium stored thereon.

The AI-based system 300 may be in communication with laboratories 322 and batch plants 323 via a computer network 324 (for example, the Internet or an intranet). As shown in FIG. 3, the computer 302 may include a network interface 326 to enable communication over the network 324. As discussed in the disclosure each laboratory 322 may generate concrete mix data 328 for communication to the AI-based system 300. Similarly, each batch plant 323 may generate concrete mix data 330 for communication to the AI-based system 300. The AI-based system 300 may obtain concrete mix data 328 and 330 and use in accordance with the techniques described herein to develop, train, and use a concrete mix AI model. Additionally, the AI-based system 300 may provide data to the laboratories 322 and batch plants 323. For example, the AI-based system 300 may provide concrete mix performance data to the laboratories 322 or provide designs for optimized concrete mixes to the batch plants 323. The laboratories 322 and batch plants 323 may optimize concrete mixes based on the performance data

Ranges may be expressed in the disclosure as from about one particular value, to about another particular value, or both. When such a range is expressed, it is to be understood that another embodiment is from the one particular value, to the other particular value, or both, along with all combinations within said range.

Further modifications and alternative embodiments of various aspects of the disclosure will be apparent to those skilled in the art in view of this description. Accordingly, this description is to be construed as illustrative only and is for the purpose of teaching those skilled in the art the general manner of carrying out the embodiments described in the disclosure. It is to be understood that the forms shown and described in the disclosure are to be taken as examples of embodiments. Elements and materials may be substituted for those illustrated and described in the disclosure, parts and processes may be reversed or omitted, and certain features may be utilized independently, all as would be apparent to one skilled in the art after having the benefit of this description. Changes may be made in the elements described in the disclosure without departing from the spirit and scope of the disclosure as described in the following claims. Headings used in the disclosure are for organizational purposes only and are not meant to be used to limit the scope of the description.

What is claimed is:

1. A method for determining a concrete mix composition, comprising:
obtaining a plurality of concrete mix compositions and respective mix properties, the respective mix properties comprising compressive strength, slump measurements, temperature measurements, tensile strength measurements, flexural strength measurements, elastic modulus measurements, Poisson's ratio measurements, creep measurements, chloride penetration measurements, permeability measurements or a combination thereof, wherein the plurality of concrete mix compositions and respective mix properties are obtained from respective laboratory tests of the plurality of concrete mix compositions, respective batch plant tests of the plurality of concrete mix compositions, or a combination thereof, wherein the plurality of concrete mix compositions comprise a production concrete mix composition and a trial concrete mix composition;
processing the plurality of concrete mix compositions and respective mix properties to obtain a training dataset and testing dataset;
training a concrete mix artificial intelligence (AI) model using the plurality of concrete mix compositions and respective mix properties of the training dataset to create a concrete mix AI model; and
using the trained concrete mix AI model to determine an optimized concrete mix composition having a specific set of mix properties.

2. The method of claim 1, comprising:
obtaining a plurality of environmental impact parameters associated with the plurality of concrete mix compositions;
processing the plurality of concrete mix compositions and environmental impact parameters into the training dataset and the testing dataset; and
training the artificial intelligence (AI) using the plurality of concrete mix compositions and environmental impact parameters of the training dataset to create the concrete mix AI model.

3. The method of claim 2, comprising using the trained concrete mix AI model to determine an environmental product declaration (EPD) of one of the plurality of concrete mix compositions or the optimized concrete mix composition.

4. The method of claim 1, wherein using the trained concrete mix AI model to determine an optimized concrete mix composition having a specific set of mix properties comprising using the trained concrete mix AI model to determine an optimized concrete mix composition optimized for a raw material, wherein the optimization for the raw material comprises environmental impact, technical performance, mix properties, or a combination thereof.

5. The method of claim 1, wherein the concrete mix AI model uses an artificial neural network (ANN), fuzzy logic, a genetic algorithm, a hybrid AI model, a genetic weighted pyramid operation tree, or a combination thereof.

6. The method of claim 1, comprising using the trained concrete mix AI model to determine a performance metric of the respective laboratory tests or the respective batch plant tests.

7. The method of claim 1, comprising using the trained concrete mix AI model to determine the quality, the production, or a combination thereof of the plurality of concrete mix compositions from the respective laboratory tests or the respective batch plant tests.

8. The method of claim 1, comprising using the trained concrete mix AI model to determine a performance metric of a technician conducting the respective laboratory tests or the respective batch plant tests.

9. The method of claim 1, comprising providing an output from the AI model to a geographic information system (GIS) to monitor use of a raw material at a location in the GIS.

10. The method of claim 1, comprising:
obtaining a plurality of experiment results, each of the experiment results associated with an experiment designed to test a raw material used in concrete mixes;
processing the plurality of experiment results into the training dataset and the testing dataset; and
training the artificial intelligence (AI) using the plurality of experiment results of the training dataset to create the concrete mix AI model.

11. The method of claim 1, comprising:
providing the optimized concrete mix composition to a concrete batch plant; and
modifying a mixing process or a concrete mix composition of the concrete batch plant to produce the optimized concrete mix composition.

12. A non-transitory computer readable storage medium comprising program instructions stored thereon for determining a concrete mix composition, the program instructions executable by a processor to perform operations comprising:
obtaining a plurality of concrete mix compositions and respective mix properties, the respective mix properties comprising compressive strength, slump measurements, temperature measurements, tensile strength measurements, flexural strength measurements, elastic modulus measurements, Poisson's ratio measurements, creep measurements, chloride penetration measurements, permeability measurements or a combination thereof, wherein the plurality of concrete mix compositions and respective mix properties are obtained from respective laboratory tests of the plurality of concrete mix compositions, respective batch plant tests of the plurality of concrete mix compositions, or a combination thereof, wherein the plurality of concrete mix compositions comprise a production concrete mix composition and a trial concrete mix composition;
processing the plurality of concrete mix compositions and respective mix properties to obtain a training dataset and testing dataset;
training a concrete mix artificial intelligence (AI) model using the plurality of concrete mix compositions and respective mix properties of the training dataset to create a concrete mix AI model; and
using the trained concrete mix AI model to determine an optimized concrete mix composition having a specific set of mix properties.

13. The non-transitory computer readable storage medium of claim 12, the operations comprising:
obtaining a plurality of environmental impact parameters associated with the plurality of concrete mix compositions;
processing the plurality of concrete mix compositions and environmental impact parameters into the training dataset and the testing dataset; and
training the artificial intelligence (AI) using the plurality of concrete mix compositions and environmental impact parameters of the training dataset to create the concrete mix AI model.

14. The non-transitory computer readable storage medium of claim 13, the operations comprising using the trained concrete mix AI model to determine an environmental product declaration (EPD) of one of the plurality of concrete mix compositions or the optimized concrete mix composition.

15. The non-transitory computer readable storage medium of claim 12, wherein using the trained concrete mix AI model to determine an optimized concrete mix composition having a specific set of mix properties comprising using the trained concrete mix AI model to determine an optimized concrete mix composition optimized for a raw material, wherein the optimization for the raw material comprises environmental impact, technical performance, mix properties, or a combination thereof.

16. The non-transitory computer readable storage medium of claim 12, wherein the concrete mix AI model uses an artificial neural network (ANN), fuzzy logic, a genetic algorithm, a hybrid AI model, a genetic weighted pyramid operation tree, or a combination thereof.

17. The non-transitory computer readable storage medium of claim 12, the operations comprising using the trained concrete mix AI model to determine a performance metric of the respective laboratory tests or the respective batch plants.

18. The non-transitory computer readable storage medium of claim 12, the operations comprising using the trained concrete mix AI model to determine the quality, the production, or a combination thereof of the plurality of concrete mix compositions from the respective laboratory tests or the respective batch plant tests.

19. The non-transitory computer readable storage medium of claim 12, the operations comprising using the trained concrete mix AI model to determine a performance metric of a technician conducting the respective laboratory tests or the respective batch plant tests.

20. The non-transitory computer readable storage medium of claim 12, the operations comprising providing an output from the AI model to a geographic information system (GIS) to monitor use of a raw material at a location in the GIS.

21. The non-transitory computer readable storage medium of claim 12, the operations comprising:
obtaining a plurality of experiment results, each of the experiment results associated with an experiment designed to test a raw material used in concrete mixes;
processing the plurality of experiment results into the training dataset and the testing dataset; and
training the artificial intelligence (AI) using the plurality of experiment results of the training dataset to create the concrete mix AI model.

22. The non-transitory computer readable storage medium of claim 12, the operations comprising:
providing the optimized concrete mix composition to a concrete batch plant; and
modifying a mixing process or a concrete mix composition of the concrete batch plant to produce the optimized concrete mix composition.

23. A system for determining a concrete mix composition, comprising:
a processor;
a non-transitory computer-readable memory accessible by the processor and having executable code stored thereon, the executable code comprising a set of instructions that causes the processor to perform operations comprising:
obtaining a plurality of concrete mix compositions and respective mix properties, the respective mix properties comprising compressive strength, slump measurements, temperature measurements, tensile strength measurements, flexural strength measurements, elastic modulus measurements, Poisson's ratio measurements, creep measurements, chloride penetration measurements, permeability measurements or a combination thereof, wherein the plurality of concrete mix compositions and respective mix properties are obtained from respective laboratory tests of the plurality of concrete mix compositions, respective batch plant tests of the plurality of concrete mix compositions, or a combination thereof, wherein the plurality of concrete mix compositions comprise a production concrete mix composition and a trial concrete mix composition;
processing the plurality of concrete mix compositions and respective mix properties to obtain a training dataset and testing dataset;
training a concrete mix artificial intelligence (AI) model using the plurality of concrete mix compositions and respective mix properties of the training dataset to create a concrete mix AI model; and
using the trained concrete mix AI model to determine an optimized concrete mix composition having a specific set of mix properties.

24. The system of claim 23, the operations comprising:
obtaining a plurality of environmental impact parameters associated with the plurality of concrete mix compositions;
processing the plurality of concrete mix compositions and environmental impact parameters into the training dataset and the testing dataset; and
training the artificial intelligence (AI) using the plurality of concrete mix compositions and environmental impact parameters of the training dataset to create the concrete mix AI model.

25. The system of claim 24, the operations comprising using the trained concrete mix AI model to determine an environmental product declaration (EPD) of one of the plurality of concrete mix compositions or the optimized concrete mix composition.

26. The system of claim 23, wherein using the trained concrete mix AI model to determine an optimized concrete mix composition having a specific set of mix properties comprising using the trained concrete mix AI model to determine an optimized concrete mix composition optimized for a raw material, wherein the optimization for the raw material comprises environmental impact, technical performance, mix properties, or a combination thereof.

27. The system of claim 23, wherein the concrete mix AI model uses an artificial neural network (ANN), fuzzy logic, a genetic algorithm, a hybrid AI model, a genetic weighted pyramid operation tree, or a combination thereof.

28. The system of claim 23, the operations comprising using the trained concrete mix AI model to determine a performance metric of the respective laboratory tests or the respective batch plants.

29. The system of claim 23, the operations comprising using the trained concrete mix AI model to determine the quality, the production, or a combination thereof of the plurality of concrete mix compositions from the respective laboratory tests or the respective batch plant tests.

30. The system of claim 23, the operations comprising using the trained concrete mix AI model to determine a performance metric of a technician conducting the respective laboratory tests or the respective batch plant tests.

31. The system of claim 23, the operations comprising providing an output from the AI model to a geographic information system (GIS) to monitor use of a raw material at a location in the GIS.

32. The system of claim 23, the operations comprising:
obtaining a plurality of experiment results, each of the experiment results associated with an experiment designed to test a raw material used in concrete mixes;
processing the plurality of experiment results into the training dataset and the testing dataset; and
training the artificial intelligence (AI) using the plurality of experiment results of the training dataset to create the concrete mix AI model.

33. The system of claim 23, the operations comprising:
providing the optimized concrete mix composition to a concrete batch plant; and
modifying a mixing process or a concrete mix composition of the concrete batch plant to produce the optimized concrete mix composition.
